(19) United States
(12) Patent Application Publication
(10) Pub. No.: US 2019/0011438 A1
(43) Pub. Date: Jan. 10, 2019

(54) BIOSENSING DEVICE AND METHOD OF FABRICATING THE SAME

(71) Applicant: NDD, INC., Gyeongsangbuk-do (KR)

(72) Inventors: Sae Young AHN, Seoul (US); Hyun Hwa KWON, Gyeongsangbuk-do (KR)

(21) Appl. No.: 16/021,197

(22) Filed: Jun. 28, 2018

(30) Foreign Application Priority Data
Jul. 4, 2017 (KR) 10-2017-0084894

(51) Int. Cl. G01N 33/543; H01L 51/10; H01L 51/00; G01N 33/68; G01N 33/53; H01L 51/05; G01N 33/58; G01N 33/566; G01N 33/92; G01N 33/548

(52) U.S. Cl. CPC G01N 33/5438; H01L 51/105; H01L 51/0093; H01L 51/0048; G01N 33/6815; H01L 51/0001; H01L 51/0512; G01N 33/581; G01N 33/566; G01N 33/92; G01N 33/548; G01N 33/5308

(57) ABSTRACT

Provided is a biosensing device including unit cells each including a source electrode and a drain electrode spaced apart from each other, a sensing membrane for forming a channel between the source and drain electrodes, a gate electrode spaced apart from the sensing membrane, and a dam structure surrounding at least parts of an edge of the sensing membrane and made of an insulator, wherein the dam structure is configured to contain a precursor solution to be solidified to generate the sensing membrane.

### TECHNICAL FIELD

The present invention relates to a biosensing device and a method of fabricating the same, and more particularly, to a biosensing device having an electrode structure, and a method of fabricating the same.

### BACKGROUND ART

Many test methods used to diagnose diseases are based on color change, fluorescence, etc. due to enzyme reaction, but currently, immunoassay methods using immune reaction between antigens and antibodies are also used. Conventional immunoassay mostly uses optical measurement methods based on catalytic reaction of enzyme and optical labeling. These methods require complicated procedures by experienced laboratory researchers, high-priced and large-sized analysis devices, and long analysis times.

### DETAILED DESCRIPTION OF THE INVENTION

### Technical Problem

The present invention provides a biosensing device and a method of fabricating the same, by which performance of a sensing membrane may be maximized, an analysis time may be reduced, and a low cost may be required. However, the scope of the present invention is not limited thereto.

### Technical Solution

According to an aspect of the present invention, there is provided a biosensing device including unit cells each including a source electrode and a drain electrode spaced apart from each other, a sensing membrane for forming a channel between the source and drain electrodes, a gate electrode spaced apart from the sensing membrane, and a dam structure surrounding at least parts of an edge of the sensing membrane and made of an insulator, wherein the dam structure is configured to contain a precursor solution to be solidified to form the sensing membrane.

The sensing membrane may include carbon nanotubes (CNT), graphene, molybdenum disulfide (MoS₂), or phosphorene.

At least parts of the dam structure perpendicular to a direction proceeding from the source electrode toward the drain electrode may be provided only on the source and drain electrodes without being provided outside the source and drain electrodes.

A width of the dam structure perpendicular to the direction proceeding from the source electrode toward the drain electrode may be less than a width of the source or drain electrode.

Each of at least parts of the dam structure parallel to a direction proceeding from the source electrode toward the drain electrode may have an end provided on the source electrode and another end provided on the drain electrode.

A length of the dam structure parallel to the direction proceeding from the source electrode toward the drain electrode may be greater than a distance between the source and drain electrodes.

A solidification density of the sensing membrane may be higher in a region adjacent to the dam structure compared to a region far apart from the dam structure.

The unit cell may further include a receptor attached onto the sensing membrane and capable of binding to a target material.

The sensing membrane may be made of a material that is variable in resistance depending on the receptor and a target material bound to the receptor.

The receptor may be attached onto the sensing membrane by a functional group, and may include at least one selected from a group consisting of enzyme-substrate, ligand, amino acid, peptide, aptamer, protein, nucleic acid, lipid, and carbohydrate.

The functional group may include at least one selected from a group consisting of an amine group, a carboxyl group, and a thiol group.

The target material may be at least one selected from the group consisting of a protein, a peptide, an aptamer, a nucleic acid, an oligosaccharide, an amino acid, a carbohydrate, a dissolved gas, a sulfur oxide gas, a nitrogen oxide gas, a residual pesticide, a heavy metal, and an environmentally harmful substance.

According to another aspect of the present invention, there is provided a method of fabricating a biosensing device, the method including a first step, for preparing a structure including a source electrode and a drain electrode spaced apart from each other, a second step, for forming a dam structure across at least a gap region between the source and drain electrodes to contact the source and drain electrodes, by using an insulator, and a third step, for forming a sensing membrane for forming a channel between the source and drain electrodes, on an inner region of the dam structure including at least a part of the gap region between the source and drain electrodes, by coating a precursor solution on the inner region of the dam structure and then solidifying the precursor solution.

The sensing membrane may include carbon nanotubes (CNT), graphene, molybdenum disulfide (MoS₂), or phosphorene.

In the second step, at least parts of the dam structure perpendicular to a direction proceeding from the source electrode toward the drain electrode may be provided only on the source and drain electrodes without being provided outside the source and drain electrodes.

In the second step, a width of the dam structure perpendicular to the direction proceeding from the source electrode toward the drain electrode may be less than a width of the source or drain electrode.

In the second step, each of at least parts of the dam structure parallel to a direction proceeding from the source electrode toward the drain electrode may have an end provided on the source electrode and another end provided on the drain electrode.

In the second step, a length of the dam structure parallel to the direction proceeding from the source electrode toward the drain electrode may be greater than a distance between the source and drain electrodes.

### Advantageous Effects

As described above, according to some embodiments of the present invention, a biosensing device and a method of fabricating the same, by which performance of a sensing membrane may be maximized, an analysis time may be reduced, and a low cost may be required, may be provided. However, the scope of the present invention is not limited to the above-described effect.

### DESCRIPTION OF THE DRAWINGS

FIG. 1 is a top view of a biosensing device and a unit cell that constitutes the biosensing device, according to an embodiment of the present invention.

FIG. 2 is a cross-sectional view of the unit cell of the biosensing device, according to an embodiment of the present invention.

FIGS. 3 and 4 are sequential top views for describing a method of fabricating a biosensing device, according to an embodiment of the present invention.

FIGS. 5 and 6 are sequential top views for describing a method of fabricating a biosensing device, according to a comparative example of the present invention.

FIG. 7 is a cross-sectional view for describing a general process of generating a sensing membrane by solidifying a precursor solution.

FIG. 8 is a top view of a biosensing device and a unit cell thereof, according to a modified embodiment of the present invention.

### MODE OF THE INVENTION

Hereinafter, the present invention will be described in detail by explaining embodiments of the invention with reference to the attached drawings. Throughout the specification, it will be understood that when an element, such as a layer, a pattern, a region, or a substrate, is referred to as being “on” another element, it may be directly on the other element or intervening elements may be present. In contrast, when an element is referred to as being “directly on” another element, there are no intervening elements present.

In the drawings, variations from the shapes of the illustrations as a result, for example, of manufacturing techniques and/or tolerances, are to be expected. Thus, the embodiments of the invention should not be construed as limited to the particular shapes of regions illustrated herein, but are to include deviations in shapes that result, for example, from manufacturing. The thicknesses or sizes of layers may be exaggerated for clarity of explanation, and like reference numerals denote like elements.

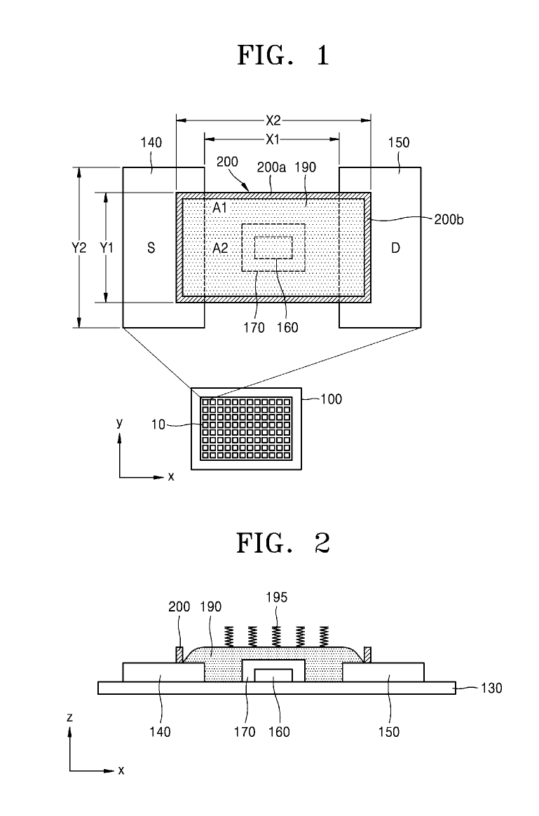

FIG. 1 is a top view of a biosensing device and a unit cell that constitutes the biosensing device, according to an embodiment of the present invention, and FIG. 2 is a cross-sectional view of the unit cell of the biosensing device, according to an embodiment of the present invention.

Referring to FIGS. 1 and 2, the biosensing device according to an embodiment of the present invention includes at least one unit cell 10 each including a source electrode 140 and a drain electrode 150 spaced apart from each other, a sensing membrane 190 that serves as a channel between the source and drain electrodes 140 and 150, a gate electrode 160 spaced apart from the sensing membrane 190, and a dam structure 200 that surrounds at least part of an edge of the sensing membrane 190 and is made of an insulator. The unit cell 10 may further include a receptor 195 attached onto the sensing membrane 190 and capable of binding to a target material. In the biosensing device according to an embodiment of the present invention, the unit cells 10 may for example be arranged in an array on a substrate 100. A lower substrate 130 illustrated in FIG. 2 may be a part of the substrate 100 illustrated in FIG. 1, or may be separately provided on the substrate 100.

The gate electrode 160 is spaced apart from the source and drain electrodes 140 and 150. The gate electrode 160 may be electrically insulated from the sensing membrane 190 by an insulating member 170 provided between the sensing membrane 190 and the gate electrode 160.

The shapes and locations of the gate electrode 160 and the insulating member 170 in FIGS. 1 and 2 are merely examples and may be variously changed. The technical idea of the present invention is not limited by specific locations and shapes of the gate electrode 160, etc.

The receptor 195 may be attached onto the sensing membrane 190 by a functional group. The receptor 195 may include at least one selected from the group consisting of, for example, an enzyme substrate, a ligand, an amino acid, a peptide, an aptamer, a protein, a nucleic acid, a lipid, and a carbohydrate. The functional group may include at least one selected from the group consisting of, for example, an amine group, a carboxyl group, and a thiol group. The target material may include at least one selected from the group consisting of, for example, a protein, an aptamer, a peptide, a nucleic acid, an oligosaccharide, an amino acid, a carbohydrate, a dissolved gas, a sulfur oxide gas, a nitrogen oxide gas, a residual pesticide, a heavy metal, and an environmentally harmful substance.

The sensing membrane 190 may be made of a material that can vary in resistance depending on the receptor 195 and a target material bound to the receptor. The material of the sensing membrane 190 may include, for example, carbon nanotubes (CNT), graphene, molybdenum disulfide (MoS₂), or phosphorene. In the biosensing device according to a modified embodiment of the present invention, the sensing membrane 190 may be made of a material that can vary in resistance by reacting directly with the above-described target material without interposing the receptor 195.

The sensing membrane 190 is formed by supplying a liquid-state precursor solution to a region including a space between the source and drain electrodes 140 and 150, and solidifying the precursor solution. The solidifying process may include at least one process selected among natural drying, heat drying, and fan drying.

The dam structure 200 made of an insulator may contain the liquid-state precursor solution supplied to form the sensing membrane 190. While the precursor solution supplied to the region including the space between the source and drain electrodes 140 and 150 is being solidified, the dam structure 200 may allow the precursor solution to stay only in a desired region and not to flow to an undesired region.

Referring to FIG. 1, in the biosensing device according to an embodiment of the present invention, at least parts of the dam structure 200 may include first structures 200a extending in a direction parallel to a direction proceeding from the source electrode 140 toward the drain electrode 150 (e.g., an x-axis direction), and each having an end provided on the source electrode 140 and the other end provided on the drain electrode 150. A length X2 of the dam structure 200 parallel to the direction proceeding from the source electrode 140 toward the drain electrode 150 may indicate a length of the first structures 200a in a direction parallel to the x-axis direction, and may be greater than a distance X1 between the source and drain electrodes 140 and 150. If the above-described condition is not satisfied, the sensing membrane 190 formed by solidifying the precursor solution may not be in contact with the source and drain electrodes 140 and 150 and thus a channel may not be formed.

In the biosensing device according to an embodiment of the present invention, at least parts of the dam structure 200 may include second structures 200b extending in a direction perpendicular to the direction proceeding from the source electrode 140 toward the drain electrode 150 (e.g., a y-axis direction), and provided only on the source and drain electrodes 140 and 150 without being provided outside the source and drain electrodes 140 and 150. For example, ends of the second structures 200b may be provided on the source and drain electrodes 140 and 150 without being provided outside the source and drain electrodes 140 and 150. A width Y1 of the dam structure 200 perpendicular to the direction proceeding from the source electrode 140 toward the drain electrode 150 (e.g., the x-axis direction) may be less than a width Y2 of the source or drain electrode 140 or 150.

In the biosensing device according to an embodiment of the present invention, the dam structure 200 may include both of the first and second structures 200a and 200b. For example, the dam structure 200 may be a rectangular structure in which a pair of first structures 200a spaced apart from each other and a pair of second structures 200b spaced part from each other are connected to each other to form a closed structure. Points where the first and second structures 200a and 200b meat each other may be located on the source and drain electrodes 140 and 150.

Unlike this, according to a modified embodiment, the dam structure 200 may include both of a pair of first structures 200a spaced apart from each other and a pair of second structures 200b spaced part from each other, and have an open structure in which the first and second structures 200a and 200b do not meet each other. Even in this case, the length X2 of the first structures 200a may be greater than the distance X1 between the source and drain electrodes 140 and 150, and the width Y1 of the second structures 200b may be less than the width Y2 of the source and drain electrodes 140 and 150.

In a biosensing device according to a modified embodiment of the present invention, the dam structure 200 may include only the first structures 200a. For example, only a pair of first structures 200a spaced apart from each other may be provided without providing the second structures 200b, and an end of each of the first structures 200a may be provided on the source electrode 140 whereas the other end thereof may be provided on the drain electrode 150. Even in this case, the length X2 of the first structures 200a may be greater than the distance X1 between the source and drain electrodes 140 and 150.

In a biosensing device according to another modified embodiment of the present invention, the dam structure 200 may include only the second structures 200b. For example, only a pair of second structures 200b spaced apart from each other may be provided without providing the first structures 200a, and one of the second structures 200b may be provided on the source electrode 140 whereas the other of the second structures 200b may be provided on the drain electrode 150. Even in this case, the width Y1 of the second structures 200b may be less than the width Y2 of the source and drain electrodes 140 and 150.

The above-described bio-sensing device according to an embodiment of the present invention may be used as a test device to diagnose a disease and may be utilized as a sensing device that uses immune reaction between an antigen and an antibody based on types of a sensing membrane and a receptor. In this case, since an electrical measurement result is used, an analysis process may not be complicated, a device for analysis may be low-priced, and a short analysis time may be taken.

The number of unit cells 10 per substrate 100 is 8×12, i.e., 96 in total, in FIG. 1 but is not limited thereto. If the size of the unit cells 10 is reduced to a nanoscale, the number of unit cells 10 may be increased to, for example, 4 times, 16 times, 64 times, 256 times, 1024 times, 4096 times, or 16384 times the value 8×12. As such, by increasing the number of unit cells per substrate, the bio-sensing device of the present invention may diagnose various diseases and greatly reduce a test time and a test cost.

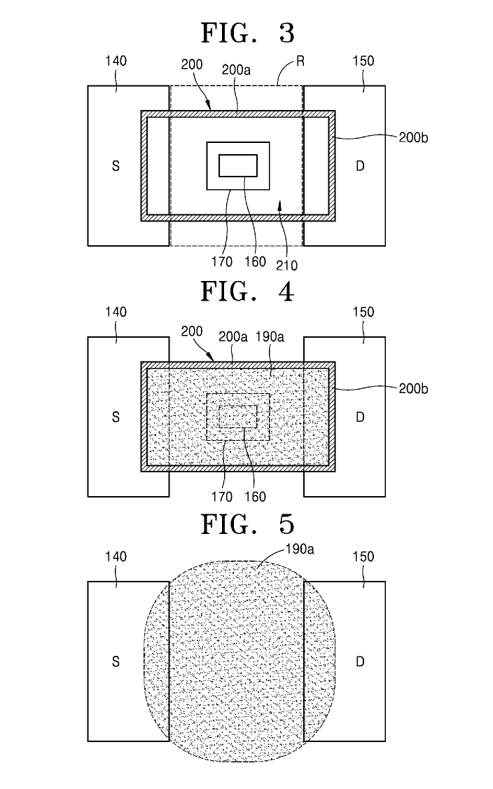

FIGS. 3 and 4 are sequential top views for describing a method of fabricating a biosensing device, according to an embodiment of the present invention.

Referring to FIG. 3, in the method of fabricating the biosensing device, according to an embodiment of the present invention, a structure including the source and drain electrodes 140 and 150 spaced apart from each other is prepared and then the dam structure 200 is formed on at least a part of a gap region R between the source and drain electrodes 140 and 150 by using an insulator. For example, the dam structure 200 may be provided across at least the gap region R between the source and drain electrodes 140 and 150 to contact the source and drain electrodes 140 and 150. The dam structure 200 may vary in shape, position, size, etc., and a detailed description thereof has been provided above in relation to FIG. 1.

Although not shown in FIGS. 3 and 4, regions where the source and drain electrodes 140 and 150 are in contact with the sensing membrane 190 may have a comb shape. According to the above configuration, bondability or connectivity between the sensing membrane 190 and the source and drain electrodes 140 and 150 may be increased.

Referring to FIG. 4, the precursor solution 190a is coated on an inner region of the dam structure 200 including at least a part of the gap region R between the source and drain electrodes 140 and 150. The precursor solution 190a may include CNT, graphene, MoS₂, or phosphorene as a solute. The inner region of the dam structure 200 may include a center part of the gap region R between the source and drain electrodes 140 and 150. The sensing membrane 190 for forming a channel between the source and drain electrodes 140 and 150 may be formed by solidifying the precursor solution 190a coated on the inner region of the dam structure 200.

The size, position, and shape of the sensing membrane 190 may be determined based on the size, position, and shape of the dam structure 200. Based on the above description of the dam structure 200, a side of the sensing membrane 190 may be provided on the source electrode 140 whereas the other side of the sensing membrane 190 may be provided on the drain electrode 150, and a length of the sensing membrane 190 may be greater than a distance between the source and drain electrodes 140 and 150 whereas a width of the sensing membrane 190 may be less than a width of the source and drain electrodes 140 and 150.

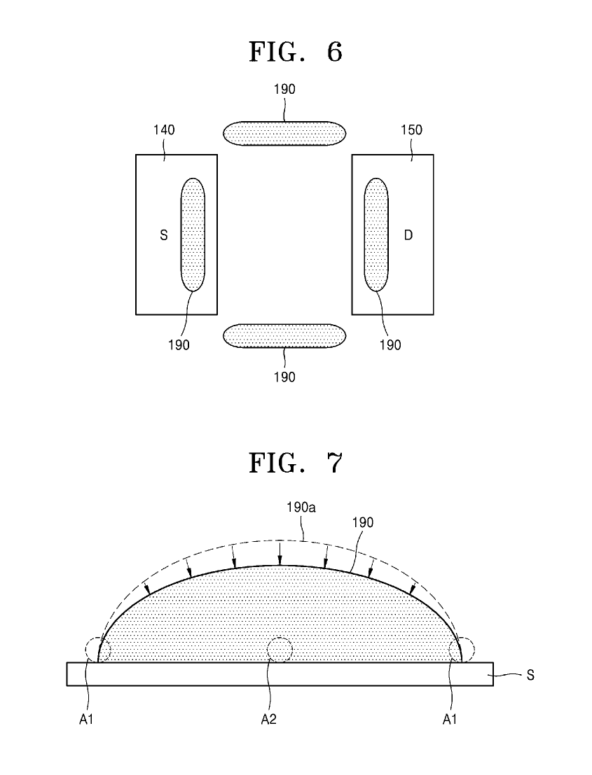

FIGS. 5 and 6 are sequential top views for describing a method of fabricating a biosensing device, according to a comparative example of the present invention.

Referring to FIGS. 5 and 6, in the method of fabricating the biosensing device, according to a comparative example of the present invention, the precursor solution 190a is coated on a gap region between the source and drain electrodes 140 and 150 without using the dam structure 200. In this case, the precursor solution 190a flows beyond the gap region between the source and drain electrodes 140 and 150. When the precursor solution 190a is solidified in this state, the sensing membrane 190 may be divided into separate pieces as illustrated in FIG. 6 without forming a channel for connecting the source and drain electrodes 140 and 150.

While the precursor solution 190a is being dried and solidified, a solute of the precursor solution 190a may be mostly concentrated in edges of the region of the precursor solution 190a illustrated in FIG. 5. Therefore, even when the sensing membrane 190 formed by solidifying the precursor solution 190a connects the source and drain electrodes 140 and 150, the solute of the precursor solution 190a may be mostly concentrated in regions corresponding to the pieces of the sensing membrane 190 illustrated in FIG. 6, and thus electrical connection between the source and drain electrodes 140 and 150 may not be good.

FIG. 7 is a cross-sectional view for describing a general process of generating the sensing membrane 190 by solidifying the precursor solution 190a.

Referring to FIG. 7, when the sensing membrane 190 is formed by solidifying the precursor solution 190a, a region A1 where the precursor solution 190a is in contact with the substrate S is constantly maintained and thus the sensing membrane 190 is also in contact with the substrate S in the region A1. Meanwhile, at least a part of a solvent included in the precursor solution 190a evaporates more at a location where the precursor solution 190a is far apart from the substrate S. Therefore, a density of a solute of the precursor solution 190a remaining in the sensing membrane 190 may be higher in the edge region A1 compared to a central region A2. This phenomenon may be easily understood based on the coffee-ring effect indicating a phenomenon that stain produced by an evaporated drop of coffee is thicker at the edge compared to the center.

Referring to FIGS. 1 and 7, a solidification density of the sensing membrane 190, which is understandable as the density of the solute of the precursor solution 190a remaining in the sensing membrane 190, may be higher in the region A1 adjacent to the dam structure 200 compared to the region A2 far apart from the dam structure 200.

Therefore, when the position, size, and shape of the dam structure 200 are controlled as described above in relation to FIG. 1, the solidification density of the sensing membrane 190 may be controlled and thus electrical conductivity of the channel formed between the source and drain electrodes 140 and 150 may be increased.

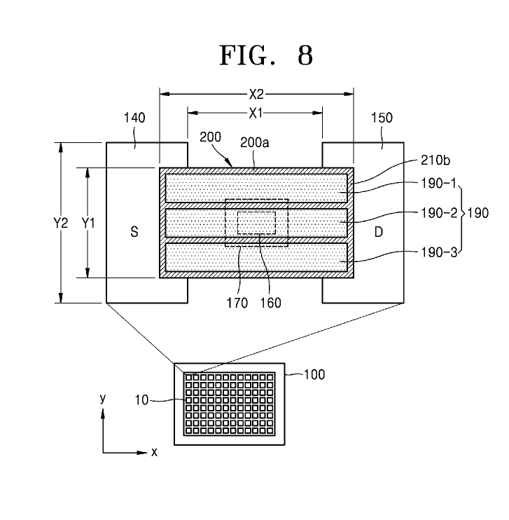

FIG. 8 is a top view of a biosensing device and a unit cell 10 thereof, according to a modified embodiment of the present invention.

Referring to FIG. 8, a dam structure 200 may include first structures 200a extending in a direction parallel to a direction proceeding from a source electrode 140 toward a drain electrode 150 (e.g., an x-axis direction), and each having an end provided on the source electrode 140 and the other end provided on the drain electrode 150, and second structures 200b extending in a direction perpendicular to the direction proceeding from the source electrode 140 toward the drain electrode 150 (e.g., a y-axis direction), and provided only on the source and drain electrodes 140 and 150 without being provided outside the source and drain electrodes 140 and 150.

As described above, a solidification density of a sensing membrane 190 may be higher in a region adjacent to the dam structure 200 compared to a region far apart from the dam structure 200. When three or more separate dam structures 200 are formed by the first structures 200a provided in the direction proceeding from the source electrode 140 toward the drain electrode 150, compared to the case of FIG. 1, electrical conductivity of a channel formed between the source and drain electrodes 140 and 150 may be further increased.

In the biosensing device illustrated in FIG. 1, the solute of the precursor solution 190a may be concentrated near the pair of first structures 200a during solidification and thus electrical channels may be dominantly formed between upper and lower parts of the source and drain electrodes 140 and 150. However, in the biosensing device illustrated in FIG. 8, even when a solute of a precursor solution 190a is concentrated near the first structures 200a during solidification, electrical channels are formed not only between upper and lower parts but also between central parts of the source and drain electrodes 140 and 150 and thus electrical conductivity may be increased.

For example, when the dam structure 200 includes four first structures 200a extending between the source and drain electrodes 140 and 150, the sensing membrane 190 formed by solidifying the precursor solution 190a may include a first sensing membrane 190-1, a second sensing membrane 190-2, and a third sensing membrane 190-3 separately formed between the four first structures 200a. In this case, since a solidification density of the second sensing membrane 190-2 is concentrated near the first structures 200a, ultimately, not only channels extending between the upper and lower parts of the source and drain electrodes 140 and 150 but also channels extending between the central parts of the source and drain electrodes 140 and 150 may be effectively formed and thus electrical conductivity may be increased.

While the present invention has been particularly shown and described with reference to embodiments thereof, it will be understood by one of ordinary skill in the art that various changes in form and details may be made therein without departing from the spirit and scope of the present invention as defined by the following claims.

1. A biosensing device comprising:
at least one unit cell, each unit cell including:
a source electrode and a drain electrode spaced apart from each other;
a sensing membrane that serves as a channel between the source electrode and the drain electrode;
a gate electrode spaced apart from the sensing membrane; and
a dam structure that surrounds at least part of an edge of the sensing membrane and is made of an insulator,
wherein the dam structure is configured to contain a precursor solution to be solidified to form the sensing membrane.

2. The biosensing device of claim 1, wherein the sensing membrane comprises carbon nanotubes (CNT), graphene, molybdenum disulfide (MoS2), or phosphorene.

3. The biosensing device of claim 1, wherein at least parts of the dam structure perpendicular to a direction proceeding from the source electrode toward the drain electrode are provided only on the source and drain electrodes without being provided outside the source and drain electrodes.

4. The biosensing device of claim 3, wherein a width of the dam structure perpendicular to the direction proceeding from the source electrode toward the drain electrode is less than a width of the source or drain electrode.

5. The biosensing device of claim 1, wherein each of at least parts of the dam structure parallel to a direction proceeding from the source electrode toward the drain electrode has an end provided on the source electrode and another end provided on the drain electrode.

6. The biosensing device of claim 5, wherein a length of the dam structure parallel to the direction proceeding from the source electrode toward the drain electrode is greater than a distance between the source and drain electrodes.

7. The biosensing device of claim 1, wherein a solidification density of the sensing membrane is higher in a region adjacent to the dam structure compared to a region far apart from the dam structure.

8. The biosensing device of claim 1, wherein the unit cell further comprises a receptor attached onto the sensing membrane and capable of binding to a target material.

9. The biosensing device of claim 8, wherein the sensing membrane is made of a material that can vary in resistance depending on the receptor and a target material bound to the receptor.

10. The biosensing device of claim 8, wherein the receptor is attached onto the sensing membrane by a functional group and is at least one selected from the group consisting of an enzyme substrate, a ligand, an amino acid, a peptide, an aptamer, a protein, a nucleic acid, a lipid, and a carbohydrate.

11. The biosensing device of claim 10, wherein the functional group is at least one selected from the group consisting of an amine group, a carboxyl group, and a thiol group.

12. The biosensing device of claim 8, wherein the target material is at least one selected from the group consisting of a protein, a peptide, an aptamer, a nucleic acid, an oligosaccharide, an amino acid, a carbohydrate, a dissolved gas, a sulfur oxide gas, a nitrogen oxide gas, a residual pesticide, a heavy metal, and an environmentally harmful substance.

13. A method of fabricating a biosensing device, the method comprising:
preparing a structure comprising a source electrode and a drain electrode spaced apart from each other;
forming a dam structure across at least a gap region between the source and drain electrodes to contact the source and drain electrodes, by using an insulator; and
forming a sensing membrane that serves as a channel between the source electrode and the drain electrode, on an inner region of the dam structure including at least a part of the gap region between the source and drain electrodes, by coating a precursor solution on the inner region of the dam structure and then solidifying the precursor solution.

14. The method of claim 13, wherein the sensing membrane comprises carbon nanotubes (CNT), graphene, molybdenum disulfide (MoS2), or phosphorene.

15. The method of claim 13, wherein at least parts of the dam structure perpendicular to a direction proceeding from the source electrode toward the drain electrode are provided only on the source and drain electrodes without being provided outside the source and drain electrodes.

16. The method of claim 15, wherein a width of the dam structure perpendicular to the direction proceeding from the source electrode toward the drain electrode is less than a width of the source or drain electrode.

17. The method of claim 13, wherein each of at least parts of the dam structure parallel to a direction proceeding from the source electrode toward the drain electrode has an end provided on the source electrode and another end provided on the drain electrode.

18. The method of claim 17, wherein a length of the dam structure parallel to the direction proceeding from the source electrode toward the drain electrode is greater than a distance between the source and drain electrodes.
